(19) United States
(12) Patent Application Publication
(10) Pub. No.: US 2023/0141034 A1
(43) Pub. Date: May 11, 2023

(54) ADAMANTANE-1-CARBONYL THIOUREA, AS P2X RECEPTOR ANTAGONIST

(71) Applicant: COMSATS UNIVERSITY ISLAMABAD, Islamabad (PK)

(72) Inventors: Jamshed IQBAL, Abbottabad (PK); Abid MAHMOOD, Abbottabad (PK)

(73) Assignee: COMSATS UNIVERSITY ISLAMABAD, Islamabad (PK)

(21) Appl. No.: 17/976,139

(22) Filed: Oct. 28, 2022

(30) Foreign Application Priority Data
Nov. 1, 2021 (PK) 802/2021

(51) Int. Cl. C07C 335/26; C07D 213/75; C07D 215/40

(52) U.S. Cl. CPC C07C 335/26; C07D 213/75; C07D 215/40; C07C 2603/74

(57) ABSTRACT

Present invention is directed to Adamantane-1-carbonyl thioureas of formula I,

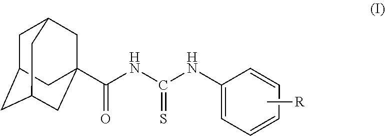

Wherein, R is selected from 2-OH, 3-OH, 4-OH, 3,5 diCl, 4-OH, 3-CF3, 5-Br, (2-Cl, pyridine-3-yl), 3-SO2NH2, 2-Cl, 5-NO2, 2-OCH3, 3-OCH3, 4-OCH3, 3,4-diOCH3, 4-Br, 2-Br,4-CH3, 2-CH3, 4-Br, Quinolin-8-yl, 2-Br-4-isopropyl, and 3-(Dimethylamino)propane-1-yl, and their use as P2X Receptor Antagonist for the treatment of inflammation, pain, osteoporosis, neurodegenerative disorders, spinal card injury, hypertension, or urinary incontinence.

### 1. BACKGROUND

Antagonists of P2X receptors have found a wide range of therapeutic applications for inflammation, pain, osteoporosis, neurodegenerative disorders, spinal cord injury, hypertension, and urinary incontinence. Some of the P2X receptor antagonists are under clinical trials for pain, rheumatoid arthritis, and cough. P2X2 receptors knockout mice exhibited reduced pain response and bladder distension reflexes. Thus, the potential inhibition of P2X2 receptors may be a therapeutic tool for the treatment of pain and urinary incontinence. Finding P2X4 receptor inhibitors might be useful for the treatment of neuropathic pain, neurodegenerative disorders, multiple sclerosis, stroke, epilepsy, and spinal cord injury. P2X7 receptor has been proved to be a potential drug target for the treatment of pain, inflammation, neurodegenerative disorders, anxiety, depression, bipolar disorders, multiple sclerosis, cerebral ischemia, brain, and spinal cord injury. Inhibition of P2X7 receptors might be very effective for the treatment of cancer, including brain cancers.

Adamantane derivatives are found to be very potent compounds and have surprising clinical efficacy from systemic to topical applications. Amantadine, Rimantadine, Memantine, Adapalene, Tromantadine, Saxagliptin, and Vildagliptin are in current clinical practice and have a wide spectrum of clinical uses (antidiabetic, antiviral, anti-inflammatory, anti-Parkinsonism, and anti-Alzheimer disease). Adamantane amides are known to be highly potent inhibitors of P2X receptors. A carboxamide linkage is present between an aryl group and a cycloalkyl group. All these derivatives were based on a series of potent carboxamides derivatives that were first reported by AstraZeneca (Guile, et al., 2009, Antagonists of the P2X7 receptor. From lead identification to drug development. Journal of medicinal chemistry, 52(10), 3123-3141). In addition, AZD9056 is the second most auspicious example that successfully progressed in phase II clinical trials, but this compound also failed due to low efficacy in various inflammatory diseases. This failure did not result to abandon adamantane core, instead, it regained interest in the adamantane scaffold as P2X7R antagonists. Abbott laboratories have filed a patent for that were found to be effective for the treatment of inflammation and neuropathic pain in rat models (Nelson, et al., 2008, Structure-activity relationship studies on N′-aryl carbohydrazide P2X7 antagonists. Journal of medicinal chemistry, 51(10), 3030-3034). Dichloropyridine-based adamantane analogue has significant activity against h-P2X7R and was studied for its anti-inflammatory activities. Thiourea derivatives were also known to possess antagonist activities towards h-P2X7R and have IC₅₀±SEM of 2.42±0.12 μM and 4.24±0.14 μM, respectively.

The present invention relates to the screening and synthesis of adamantane thiourea derivatives for inhibitory potential towards h-P2X2, h-P2X4, h-P2X5, and h-P2X7 receptors. Here has been combined different structural components of known inhibitors, to potentiate the activities of resulting synthesized derivatives.

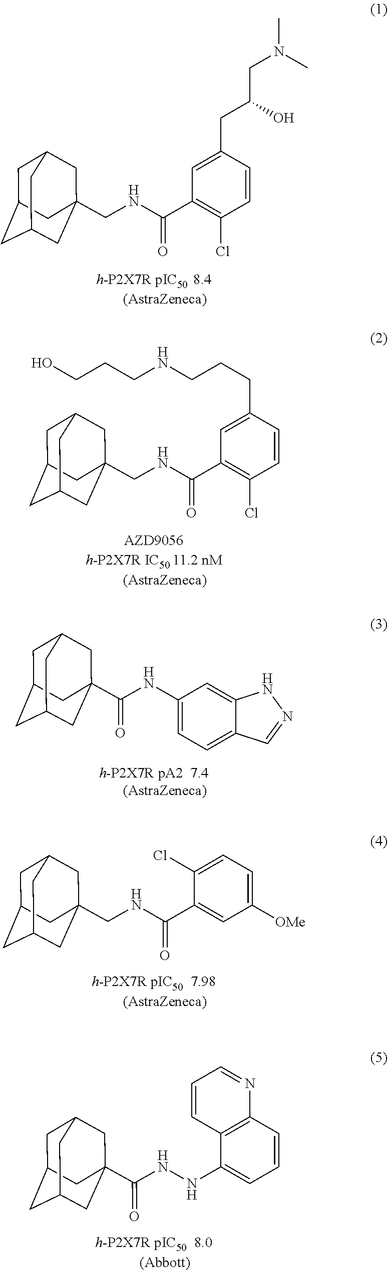 [SMILES: CN(C)C[C@H](O)Cc1ccc(Cl)c(C(=O)NCC23CC4CC(CC(C4)C2)C3)c1 ; O=C(NCC12CC3CC(CC(C3)C1)C2)c1cc(CCCNCCCO)ccc1Cl ; O=C(Nc1ccc2cn[nH]c2c1)C12CC3CC(CC(C3)C1)C2 ; COc1ccc(Cl)c(C(=O)NCC23CC4CC(CC(C4)C2)C3)c1 ; O=C(NNc1cccc2ncccc12)C12CC3CC(CC(C3)C1)C2]

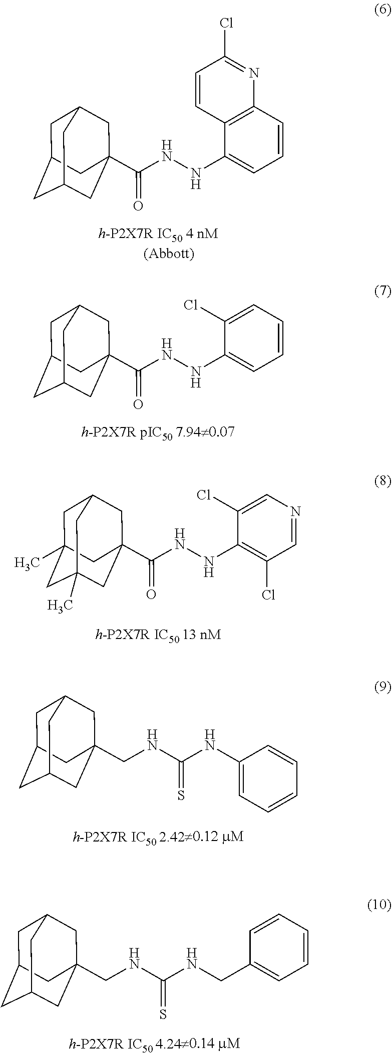 [SMILES: O=C(NNc1cccc2nc(Cl)ccc12)C12CC3CC(CC(C3)C1)C2 ; O=C(NNc1ccccc1Cl)C12CC3CC(CC(C3)C1)C2 ; CC12CC3CC(C)(C1)CC(C(=O)NNc1c(Cl)cncc1Cl)(C3)C2 ; S=C(NCC12CC3CC(CC(C3)C1)C2)Nc1ccccc1 ; S=C(NCc1ccccc1)NCC12CC3CC(CC(C3)C1)C2]

Some reported antagonists of h-P2X7 Receptor including the patent compounds filed by AstraZeneca and Abbott Laboratories.

### 2. BRIEF DESCRIPTION OF DRAWINGS

FIG. 1a. Mechanism of receptor inhibition by potent inhibitors of h-P2X4.

FIG. 1b. Mechanism of receptor inhibition by potent inhibitors of h-P2X7R.

FIG. 2a. Ethidium Bromide Dye uptake assay for potent inhibitors of h-P2X7R, representing the inhibition of Io and If.

FIG. 2b. Ethidium Bromide Dye uptake assay for potent inhibitors of h-P2X7R, (a) representing the serial dilution curves for Io and If to determine the IC50 values.

### 3. Physiological Functions of P2X Receptors

P2X receptors are to be involved in various physiological processes in the body, some of their functions are listed below receptor subtypes wise.

Physiological Role P2X1 receptors:
i. Smooth Muscle Contraction
ii. Renal Autoregulation
iii. Platelet Activation

Physiological Role P2X2 receptors:
i. Regulation of Neurotransmitter release in hippocampus
ii. Nociceptive signaling
iii. Intestinal Neurotransmission
iv. Carotid body function (O₂, CO₂, pH of blood)
v. Sensory neurotransmission in Urinary bladder
vi. Gustatory signaling (Taste sensing)
vii. Endplate formation

Physiological Role P2X3 receptors:
i. Regulation of Synaptic plasticity
ii. Nociceptive signaling
iii. Intestinal Neurotransmission
iv. Sensory Neurotransmission in Urinary bladder
v. Thermal sensation
vi. Gustatory signaling (Taste sensing)

Physiological Role P2X4 receptors:
i. Regulation of synaptic plasticity
ii. Modulation of chronic pain
iii. Regulation of vascular tone
iv. Control of contractility of the
v. Cardiomyocytes

Physiological Role P2X7 receptors:
i. Pro-inflammatory cytokine release
ii. Immune cells activation
iii. Bone metabolism
iv. Regulation of exocrine gland secretion

### 4. Therapeutic Potential of P2X Receptors Antagonists

An overview of the importance of P2X Receptor antagonists is listed below that are believed to be novel targets for the treatment of various diseases, more likely neurodegenerative disorders, neuropathic pain, inflammations, depression, etc. Selective and potent antagonists have the following therapeutic outcomes.

Inhibition of P2X1 receptors is a novel approach for the treatment of neuroprotective action, treatment of Parkinson's disease, and prevention of Heart Stroke and Thrombosis.

Inhibition of P2X2 receptors, result to relieve pain and neuroprotective action.

Inhibition of P2X4 receptors leads to Relieve Neuropathic Pain, Treatment of Epilepsy, Multiple sclerosis, Stroke, and Spinal Cord Injury. Treatment of neurodegenerative diseases i.e., Alzheimer's and Parkinson's disease.

Inhibition of P2X7 receptor is an emerging drug target for the treatment of inflammatory diseases, pain (nociceptive, inflammatory, neuropathic, chronic), treatment of Multiple sclerosis, Cerebral ischemia, Spinal cord, and Brain injury. Relieve Neurodegenerative disorders, Depression, Bipolar disorders, and Anxiety.

### 5. Description of the Invention

The invention relates to a compound of formula I,

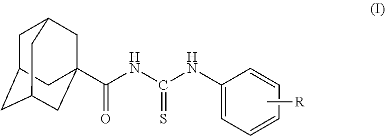

Here, the R group represents the variety of substitutions, these include hydrophilic substitutions such as hydroxy, and methoxy groups substitutions. Sulfonamide substitutions were also introduced to enhance the potency and partition coefficient of synthesized derivatives. Various halogens and nitro substitutions were also added to aryl moiety. Heterocyclic moieties like pyridine and quinoline were included which confer the highest potency among the synthesized derivatives.

The following moieties were attached at the R substitutions.

2-OH, 3-OH, 4-OH, (3,5 diCl, 4-OH), (3-CF₃, 5-Br), (2-Cl, pyridine-3-yl), 3-SO₂NH₂, (2-Cl, 5-NO₂), 2-OCH₃, 3-OCH₃,4-OCH₃, (3,4-diOCH₃), 4-Br, (2-Br,4-CH₃), (2-CH₃, 4-Br), Quinolin-8-yl, (2-Br-4-isopropyl), and [3-(Dimethylamino)propane-1-yl].

Following synthesized derivatives, listed below are potent and selective inhibitors of h-P2X2, 4, 5, and 7 receptors. These antagonists have diverse therapeutic applications and have a nontoxic profile.

1. N-((2-hydroxyphenyl)carbamothioyl)adamantane-1-carboxamide (Ia)

2. N((3-hydroxyphenyl)carbamothioyl)adamantane-1-carboxamide (Ib)

3. N-((4-hydroxyphenyl)carbamothioyl)adamantane-1-carboxamide (Ic)

4. N-((3,5-dichloro-4-hydroxyphenyl)carbamothioyl)adamantane-1-carboxamide (Id)

5. N-((3-bromo-4-hydroxy-5-(trifluoromethyl)phenyl)carbamothioyl)adamantane-1-carboxamide (Ie)

6. N-((2-chloropyridin-3-yl)carbamothioyl)adamantane-1-carboxamide (If)

7. N-((3-sulfamoylphenyl)carbamothioyl)adamantane-1-carboxamide (Ig)

8. N-((2-chloro-5-nitrophenyl)carbamothioyl)adamantane-1-carboxamide (Ih)

9. N-((3-methoxyphenyl)carbamothioyl)adamantane-1-carboxamide (Ii)

10. N-((4-methoxyphenyl)carbamothioyl)adamantane-1-carboxamide (Ij)

11. N-((3,4-dimethoxyphenyl)carbamothioyl)adamantane-1-carboxamide (Ik)

12. N-((4-bromophenyl)carbamothioyl)adamantane-1-carboxamide (Il)

13. N-((2-bromo-4-methylphenyl)carbamothioyl)adamantane-1-carboxamide (Im)

14. N-((4-bromo-2-methylphenyl)carbamothioyl)adamantane-1-carboxamide (In)

15. N-(quinolin-8-ylcarbamothioyl)adamantane-1-carboxamide (Io)

16. N-((2-bromo-4-isopropylphenyl)carbamothioyl)adamantane-1-carboxamide (Ip)

17. N-((2,5-dibromophenyl)carbamothioyl)adamantane-1-carboxamide (Iq)

18. N-((3-(dimethylamino)propyl)carbamothioyl)adamantane-1-carboxamide (Ir)

TABLE 1
Structures of synthesized adamantane thioureas (Ia-Ir)

| Sr | | | |
|---|---|---|---|
| # | Code | Structure | Chemical Name |
| 1 | Ia | 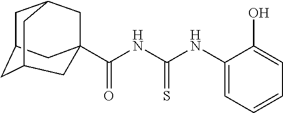 [SMILES: O=C(NC(=S)Nc1ccccc1O)C12CC3CC(CC(C3)C1)C2] | N-((2-hydroxyphenyl)carbamothioyl)adamantane-1- carboxamide |
| 2 | Ib | 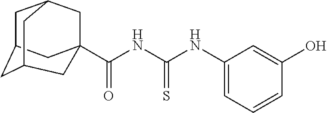 [SMILES: O=C(NC(=S)Nc1cccc(O)c1)C12CC3CC(CC(C3)C1)C2] | N-((3-hydroxyphenyl)carbamothioyl)adamantane-1- carboxamide |
| 3 | Ic | 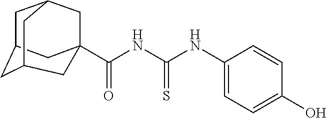 [SMILES: O=C(NC(=S)Nc1ccc(O)cc1)C12CC3CC(CC(C3)C1)C2] | N-((4-hydroxyphenyl)carbamothioyl)adamantane-1- carboxamide |
| 4 | Id | 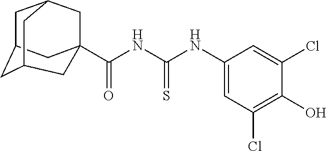 [SMILES: O=C(NC(=S)Nc1cc(Cl)c(O)c(Cl)c1)C12CC3CC(CC(C3)C1)C2] | N-((3,5-dichloro-4- hydroxyphenyl)carbamothioyl)adamantane-1- carboxamide |
| 5 | Ie | 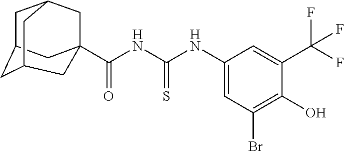 [SMILES: O=C(NC(=S)Nc1cc(Br)c(O)c(C(F)(F)F)c1)C12CC3CC(CC(C3)C1)C2] | N-((3-bromo-4-hydroxy-5- (trifluoromethyl)phenyl)carbamothioyl)adamantane- 1-carboxamide |
| 6 | If | 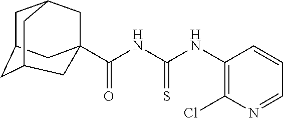 [SMILES: O=C(NC(=S)Nc1cccnc1Cl)C12CC3CC(CC(C3)C1)C2] | N-((2-chloropyridin-3- yl)carbamothioyl)adamantane-1-carboxamide |
| 7 | Ig | 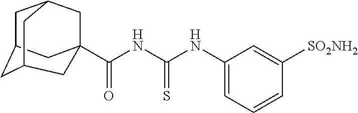 [SMILES: NS(=O)(=O)c1cccc(NC(=S)NC(=O)C23CC4CC(CC(C4)C2)C3)c1] | N-((3-sulfamoylphenyl)carbamothioyl)adamantane- 1-carboxamide |
| 8 | Ih | 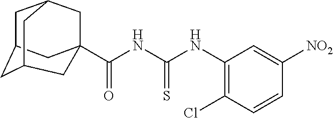 [SMILES: O=C(NC(=S)Nc1cc([N+](=O)[O-])ccc1Cl)C12CC3CC(CC(C3)C1)C2] | N-((2-chloro-5- nitrophenyl)carbamothioyl)adamantane-1- carboxamide |
| 9 | Ii | 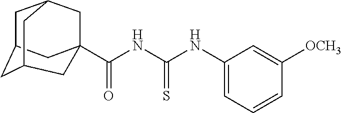 [SMILES: COc1cccc(NC(=S)NC(=O)C23CC4CC(CC(C4)C2)C3)c1] | N-((3-methoxyphenyl)carbamothioyl)adamantane- 1-carboxamide |
| 10 | Ij | 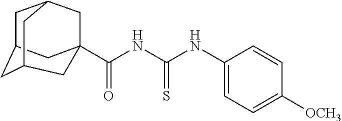 [SMILES: COc1ccc(NC(=S)NC(=O)C23CC4CC(CC(C4)C2)C3)cc1] | N-((4-methoxyphenyl)carbamothioyl)adamantane- 1-carboxamide |
| 11 | Ik | 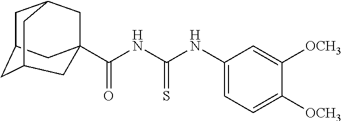 [SMILES: COc1ccc(NC(=S)NC(=O)C23CC4CC(CC(C4)C2)C3)cc1OC] | N-((3,4- dimethoxyphenyl)carbamothioyl)adamantane-1- carboxamide |
| 12 | Il | 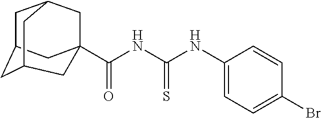 [SMILES: O=C(NC(=S)Nc1ccc(Br)cc1)C12CC3CC(CC(C3)C1)C2] | N-((4-bromophenyl)carbamothioyl)adamantane-1- carboxamide |
| 13 | Im | 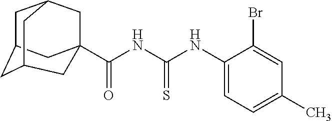 [SMILES: Cc1ccc(NC(=S)NC(=O)C23CC4CC(CC(C4)C2)C3)c(Br)c1] | N-((2-bromo-4- methylphenyl)carbamothioyl)adamantane-1- carboxamide |
| 14 | In | 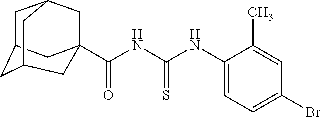 [SMILES: Cc1cc(Br)ccc1NC(=S)NC(=O)C12CC3CC(CC(C3)C1)C2] | N-((4-bromo-2- methylphenyl)carbamothioyl)adamantane-1- carboxamide |
| 15 | Io | 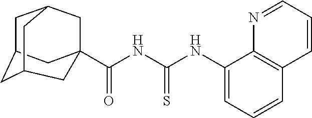 [SMILES: O=C(NC(=S)Nc1cccc2cccnc12)C12CC3CC(CC(C3)C1)C2] | N-(quinolin-8-ylcarbamothioyl)adamantane-1- carboxamide |
| 16 | Ip | 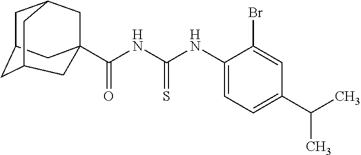 [SMILES: CC(C)c1ccc(NC(=S)NC(=O)C23CC4CC(CC(C4)C2)C3)c(Br)c1] | N-((2-bromo-4- isopropylphenyl)carbamothioyl)adamantane-1- carboxamide |
| 17 | Iq | 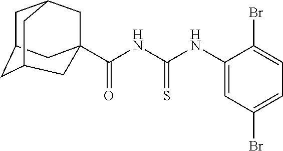 [SMILES: O=C(NC(=S)Nc1cc(Br)ccc1Br)C12CC3CC(CC(C3)C1)C2] | N-((2,5-dibromophenyl)carbamothioyl)adamantane- 1-carboxamide |
| 18 | Ir | 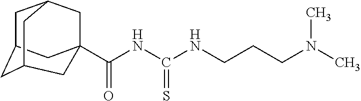 [SMILES: CN(C)CCCNC(=S)NC(=O)C12CC3CC(CC(C3)C1)C2] | N-((3- (dimethylamino)propyl)carbamothioyl)adamantane- 1-carboxamide |

a. Synthesis of Adamantane Thioureas (Ia-Ir)

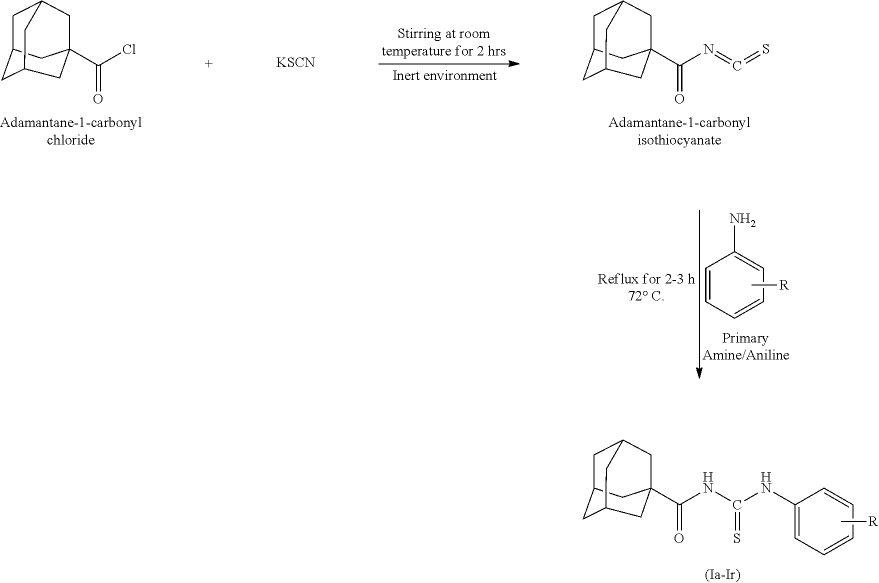

Adamantane thiourea derivatives were synthesized and obtained in very good yield. Structure elucidation was done through FTIR, ¹H NMR, ¹³C NMR, HRMS and Elemental analysis. Physical properties were also noted including color and melting of compounds.

b. Safety Profiling of Synthesized Derivatives

Cellular safety profiling of adamantane thioureas were done through MTT assay. All compounds were analyzed for toxicity at final concentration of 50 μM. None of synthesized compound exhibited cell toxicity at this concentration. Toxicity was determined against two cancer cell lines including human 1321N1 Astrocytoma cell line, HeLa cell line and a normal HEK-293 cell line.

TABLE 2
Cytotoxicity assay for the synthesized compounds against human 1321N1
astrocytoma cell line, HEK-293 cell line and HeLa cell line.

| | Human 1321N1 | HEK-293 | HeLa |
|---|---|---|---|
| | Astrocytoma Cell Line | Cell Line | Cell Line |
| | (% cyto- | (% cyto- | (% cyto- |
| Compounds | toxicity) | toxicity) | toxicity) |
| Ia | 10.33 | 26.62 | 13.06 |
| Ib | 5.67 | 6.95 | 11.03 |
| Ic | 19.00 | 3.02 | 8.66 |
| Id | 6.18 | 1.06 | 5.40 |
| Ie | 5.37 | 6.85 | 11.87 |
| If | 11.40 | 4.95 | 5.21 |
| Ig | 12.92 | 3.77 | 12.50 |
| Ih | 20.47 | 24.56 | 8.66 |
| Ii | 6.53 | 20.67 | 18.09 |
| Ij | 4.56 | 10.60 | 8.01 |
| Ik | 19.45 | 7.20 | 14.29 |
| Il | 15.40 | 18.67 | 7.19 |
| Im | 7.29 | 18.90 | 15.43 |
| In | 14.29 | 14.02 | 4.10 |
| Io | 9.12 | 17.55 | 6.22 |
| Ip | 19.74 | 20.63 | 23.51 |
| Iq | 21.19 | 14.84 | 20.74 |
| Ir | 10.05 | 5.16 | 8.14 |
| PPADS | 19.02 | 42.58 | 17.92 |
| Suramin | 18.61 | 48.01 | 21.66 |
| Reactive | 20.82 | 39.89 | 14.47 |
| Blue 2 | | | |
| BX-430 | 20.21 | 2.85 | 9.12 |
| Cisplatin | 84.75 | 86.11 | 85.01 |

c. Inhibitory Potential of Synthesized Adamantane Thiourea Derivatives: (Ia-Ir)

Inhibitory potential of adamantane thiourea derivatives towards h-P2X receptors was analyzed through Ca²⁺ flux assay. Here, Suramin, PPADS, Reactive Blue 2 and BX-430 were used as positive control. Compounds were initially analyzed at a final concentration of about 50 μM and percentage of inhibition was calculated. The compounds exhibiting percentage of inhibition more than 50%, were further investigated for serial dilution curve response. IC₅₀±SEM values were determined that were comparable with the standard inhibitors and there was an additional advantage of these synthesized inhibitors that they were selective inhibitors which is the key requirement for a drug to avoid toxicity problems.

TABLE 3
Results of Ca²⁺ flux assay to access the inhibitory
potential of h-P2X2, h-P2X4, h-P2X5, and h-P2X7R.

| | h-P2X2 | h-P2X4 | h-P2X5 | h-P2X7 |
|---|---|---|---|---|
| | IC₅₀ ± | IC₅₀ ± | IC₅₀ ± | IC₅₀ ± |
| | SEM^{a}(μM)/ | SEM^{a}(μM)/ | SEM^{a}(μM)/ | SEM^{a}(μM)/ |
| | % Inhibition | % Inhibition | % Inhibition | % Inhibition |
| Ia | 32.61 | 1.084 ± 0.02 | 40.61 | 1.474 ± 0.09 |
| Ib | 32.56 | 36.66 | 43.65 | 19.11 |
| Ic | 37.85 | 32.37 | 33.20 | 1.395 ± 0.18 |
| Id | 1.061 ± 0.05 | 1.282 ± 0.11 | 3.399 ± 0.40 | 2.453 ± 0.70 |
| Ie | 30.51 | 4.530 ± 0.39 | 20.43 | 4.024 ± 0.64 |
| If | 13.15 | 1.274 ± 0.11 | 25.53 | 0.099 ± 0.07 |
| Ig | 18.13 | 4.187 ± 0.23 | 42.50 | 0.829 ± 0.12 |
| Ih | 4.576 ± 0.21 | 1.290 ± 0.09 | 32.14 | 2.530 ± 0.72 |
| Ii | 2.306 ± 0.31 | 37.97 | 43.99 | 15.12 |
| Ij | 5.242 ± 0.32 | 44.91 | 44.52 | 29.35 |
| Ik | 0.732 ± 0.01 | 3.461 ± 0.40 | 1.481 ± 0.23 | 4.896 ± 1.04 |
| Il | 36.04 | 46.83 | 47.68 | 8.801 ± 1.12 |
| Im | 4.852 ± 0.32 | 1.562 ± 0.21 | 6.037 ± 1.20 | 3.707 ± 0.78 |
| In | 22.68 | 34.86 | 27.12 | 18.80 |
| Io | 15.32 | 25.13 | 31.54 | 0.073 ± 0.04 |
| Ip | 30.17 | 0.040 ± 0.01 | 12.41 | 42.55 |
| Iq | 41.08 | 5.730 ± 0.42 | 16.22 | 11.17 |
| Ir | 2.15 | 0.00 | 11.25 | 5.01 |
| Suramin | 13.45 ± 0.11 | 5.64 ± 0.04 | 16.07 ± 0.5 | 33.21 ± 0.47 |
| PPADS | 1.40 ± 0.10 | 5.25 ± 0.30 | 7.01 ± 0.12 | 34.27 |
| Reactive | 0.89 ± 0.41 | n.d | n.d | 2.79 ± 0.22 |
| Blue 2 | | | | |
| BX-430 | n.d | 0.874 ± 0.09 | n.d | n.d |

^{a}n = 3 unless otherwise noted,
^{b} percent inhibition at 50 μM test compound,
^{n.d} not determined

d. Mode of Receptor Inhibition

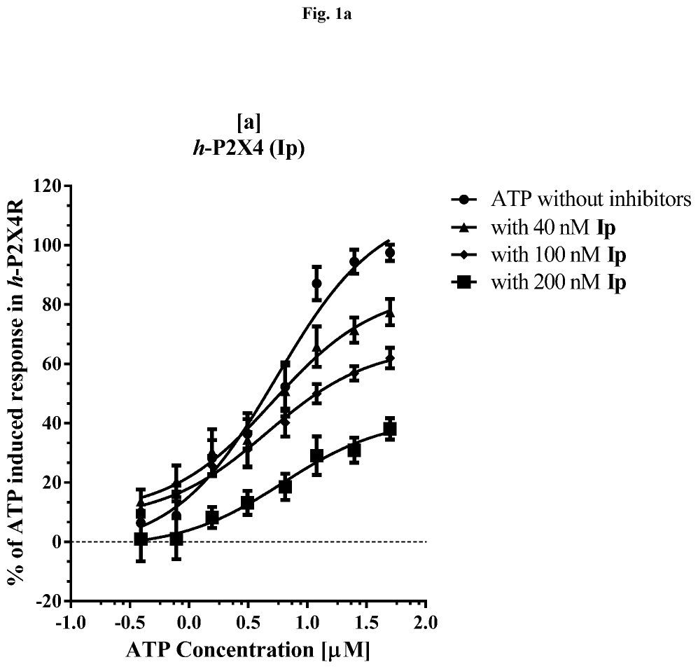

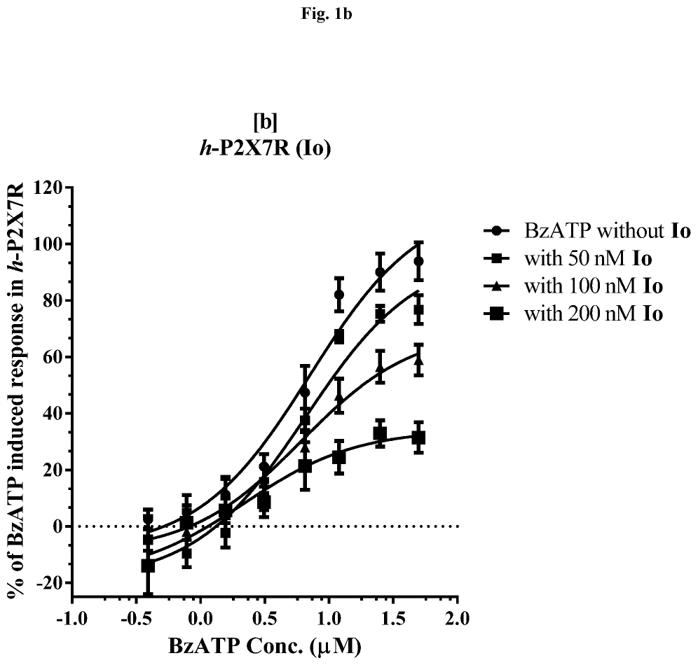

The most potent compounds Ip and Io were selected to find the mechanism of receptor inhibition. Different dilutions of compounds were administered to receptors expressed cell line. It is evident from the graphs in FIG. 1 and Table 4, that Ip and Io exhibited negative allosteric modulators against h-P2X4R and h-P2X7R, respectively.

TABLE 4
EC₅₀ value and Eₘₐₓ for ATP/BzATP induced response in the absence
and presence of different concentrations of inhibitor

| ATP/BzATP dose-response curve | | EC₅₀ (μM) | Eₘₐₓ (%) |
|---|---|---|---|
| h-P2X4R | ATP without inhibitor | 5.453 | 100 |
| | 40 nM of Ip | 5.090 | 77 |
| | 100 nM of Ip | 4.557 | 62 |
| | 200 nM of Ip | 6.187 | 38 |
| h-P2X7R | BzATP without inhibitor | 7.090 | 100 |
| | 50 nM of Io | 6.617 | 78 |
| | 100 nM of Io | 6.084 | 59 |
| | 200 nM of Io | 2.192 | 31 |

e. Ethidium Bromide Dye-Uptake Assay for P2X7 Receptors: (Pore Formation)

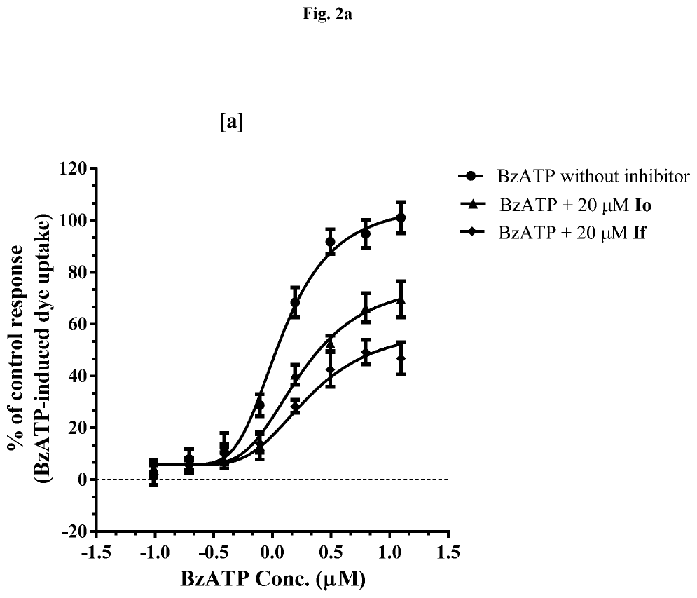

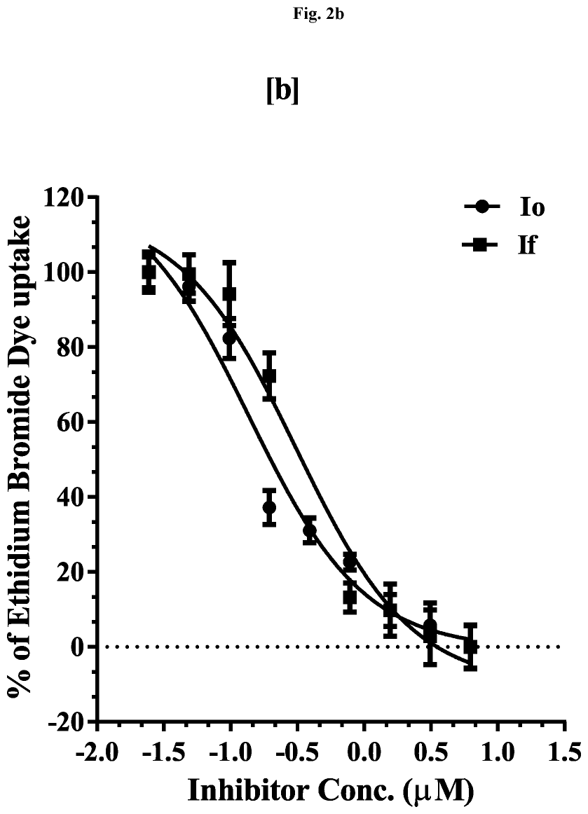

This is a specific assay to access the activities of P2X7R. Prolong exposure of P2X7R leads to pore formation that results intake of large molecular weight dye through this pore. An inhibitor of P2X7R inhibits this pore formation that confirms the inhibitory potential of compounds. Most potent inhibitors of P2X7R were selected for this assay, and serial dilution curves were also plotted to find IC₅₀±SEM values of Io and If. Results are shown in FIG. 2 and Table 5.

TABLE 5
Potency determination through BzATP induced
Ethidium Bromide dye uptake for h-P2X7R

| BzATP induced Ethidium | Eₘₐₓ % (at 20 μM | IC₅₀ ± SEM |
|---|---|---|
| Bromide dyeuptake | inhibitor concentration) | (μM) |
| BzATP without inhibitor | 100 | |
| Io | 70 | 0.136 ± 0.02 |
| If | 47 | 0.310 ± 0.07 |

f. Drug Like Properties of Adamantane Thiourea Derivatives Determines through Swiss-ADME:

Pharmacokinetic as well as physicochemical properties of Adamantane Thiourea derivatives were determined through an online in silico program, Swiss ADME launched by the Swiss Institute of Bioinformatics. Most of the synthesized compounds fulfill the drug-able criteria.

TABLE 6
Proposed Pharmacokinetic and physicochemical properties of Adamantane Thiourea derivatives

| Codes | TPSA^{a} | Lipinski violation | PAINS^{b} | WLOGP^{c} | NRB^{d} | HBD^{e} | GI Absorption | HBA^{g} |
|---|---|---|---|---|---|---|---|---|
| Ia | 93.45 | 0 | 0 | 3.23 | 5 | 3 | High | 2 |
| Ib | 93.45 | 0 | 0 | 3.23 | 5 | 3 | High | 2 |
| Ic | 93.45 | 0 | 0 | 3.23 | 5 | 3 | High | 2 |
| Id | 93.45 | 0 | 0 | 4.54 | 5 | 3 | High | 2 |
| Ie | 93.45 | 0 | 0 | 6.16 | 6 | 3 | Low | 5 |
| If | 89.11 | 0 | 0 | 3.57 | 5 | 2 | High | 2 |
| Ig | 141.76 | 0 | 0 | 3.25 | 6 | 3 | Low | 4 |
| Ih | 119.04 | 0 | 0 | 4.09 | 6 | 2 | High | 3 |
| Ii | 82.45 | 0 | 0 | 3.53 | 6 | 2 | High | 2 |
| Ij | 82.45 | 0 | 0 | 3.53 | 6 | 2 | High | 2 |
| Ik | 91.68 | 0 | 0 | 4.18 | 7 | 2 | High | 3 |
| Il | 73.22 | 0 | 0 | 4.29 | 5 | 2 | High | 1 |
| Im | 73.22 | 0 | 0 | 4.60 | 5 | 2 | High | 1 |
| In | 73.22 | 0 | 0 | 4.60 | 5 | 2 | High | 1 |
| Io | 86.11 | 0 | 0 | 4.07 | 5 | 2 | High | 2 |
| Ip | 73.22 | 0 | 0 | 4.41 | 6 | 2 | High | 1 |
| Iq | 73.22 | 0 | 0 | 5.05 | 5 | 2 | High | 1 |
| Ir | 76.46 | 0 | 0 | 2.15 | 8 | 2 | High | 2 |

^{a}Topological polar surface area,
^{b}Pan-assay interference,
^{c}Logarithm of partition coefficient between n-octanol and water,
^{d}number of rotatable bond,
^{e}hydrogen bond donor,
^{f}gastrointestinal absorption,
^{g}Hydrogen bond acceptor.

### 6. Methodology

a. Synthesis of Thiourea Derivatives as P2X Receptors Antagonists

Carboxylic acid functional group of the parent nucleus will be modified into carbonyl chloride by using thionyl chloride as a catalyst. In the subsequent reaction, potassium thiocyanate will be added to the above reaction mixture with continuous stirring at room temperature for one hour. This chemical reaction will synthesize isothiocyanates, as an important intermediate of the reaction. Primary amines will be added to isothiocyanate mixture and will be stirred for 24 h at room temperature to get the thiourea derivatives. The reaction mixture will be poured into ice-cold water that will help to precipitate the product. Then precipitates will be filtered, washed with cold water, and dried in a vacuum desiccator.

b. Calcium Mobilization Assay to Measure P2X Receptors Response Cell Culture: P2X receptors functions can be analyzed from ATP-induced increase in cytosolic Ca²⁺ concentration. Two Ca²⁺ chelating dyes, Fura-2 AM and Fluo-4 AM can be used to determine the intracellular Ca²⁺ concentration through a fluorescent imaging micro-titer plate reader (BMG Labtech: FLUOstar Galaxy®). Non-transfected human 1321N1 astrocytoma cells, as well as P2X receptors transfected human 1321N1 astrocytoma cells will be cultured in T-75 cm² cell culture flask having appropriate cell culture growth media; Dulbecco's Modified Eagle's medium (DMEM) supplemented with 10% fetal bovine serum (FBS), 75 μg/mL of Hygromycin B or human P2X receptors transfected cell lines and 1% pen-strep (antibiotics). Cells will be incubated at 37° C. in 5% CO₂ incubator until 80 to 90% cell confluency will be achieved. Cells will be maintained in an exponential growth phase throughout the experiment and closely monitored for contamination.

c. Cell Viability Assay (MTT Assay)

Human astrocytoma 1321N1 cells will be seeded in triplicate format at a density of 20,000 cells/well in 96 wells plate. After 24 h of incubation at 37° C. in CO₂ incubator, culture media will be replaced with serum-free media along with test compounds and re-incubate. After 24 h, media will be removed and add 100 μL of MTT reagent (0.5-5 mg/mL) and will be incubated for a further 4 h, at 37° C. Absorbance will be measured at 570 and 630 nm to calculate cell viability.

d. Measurement of Ca²⁺ Flux

When 1321N1 cells will reach 80 to 90% of confluency, cells will be harvested from T-75 cm² flask by trypsinization. After washing the cells with phosphate buffer saline (PBS), cells will be seeded at a density of 20,000 cells/well in black, clear bottom, poly-D-lysine coated 96 well plate. After 16 to 24 h, cell culture media will be removed and add Hank's balanced salt solution (HBSS) containing 3 μM Ca²⁺ chelating dyes Fura-2 AM or Fluo-4 AM. After 1 h of incubation at 23° C., the loading dye solution will be removed, and added seven to eight dilutions of test compounds in triplicate format. ATP (at a concentration value of EC₈₀) or buffer will be added and measured the excitation and emission at 340/520 nm and 380/520 nm through FLUOStar Galaxy®. Assay volume for the experiment will be 200 μL. IC₅₀ and EC₅₀ values will be calculated by PRISM 5.0 (GrapgPad, San Diego, Calif., USA) through non-linear regression analysis.

e. Ethidium Bromide Dye-Uptake Assay for P2X7 Receptors: (Pore Formation)

Human astrocytoma 1321N1 cells stably expressing P2X7 receptors will be seeded in the black wall, clear bottom and poly-D-lysine coated 96 wells plate at a cell density of 20,000 cells/well. After 24 h of incubation, culture medium will be removed, and wash the cells with dye uptake buffer. Cells will be treated with ethidium bromide with the subsequent addition of different concentrations of Bz-ATP to 96 wells plate in triplicate format. After 30 min of incubation at 37° C., ethidium bromide uptake will be measured through a fluorescence imaging mircoplate reader (BMG-Omega FLUOstar®) with excitation-emission filters of 525/610 nm. For screening of P2X7 receptor antagonists, cells will be treated with different concentrations of test compounds for 15 min at 37° C. before the addition of Bz-ATP.

We claim:

1. A compound of formula (I) 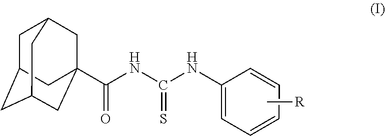
Wherein, R is selected from
2-OH,
3-OH,
4-OH,
3,5 diCl, 4-OH,
3-CF₃, 5-Br,
(2-Cl, pyridine-3-yl),
3-SO₂NH₂,
2-Cl, 5-NO₂,
2-OCH₃,
3-OCH₃,
4-OCH₃,
3,4-diOCH₃,
4-Br,
2-Br,4-CH₃,
2-CH₃, 4-Br,
Quinolin-8-yl,
2-Br-4-isopropyl, and
3-(Dimethylamino)propane-1-yl.

2. The compound of claim 1, wherein the compound is

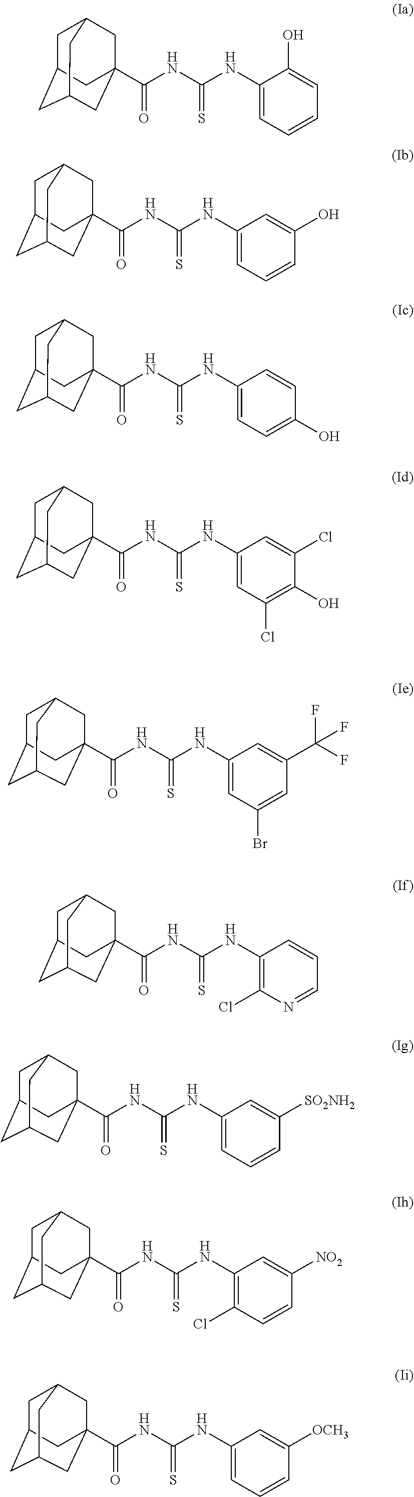

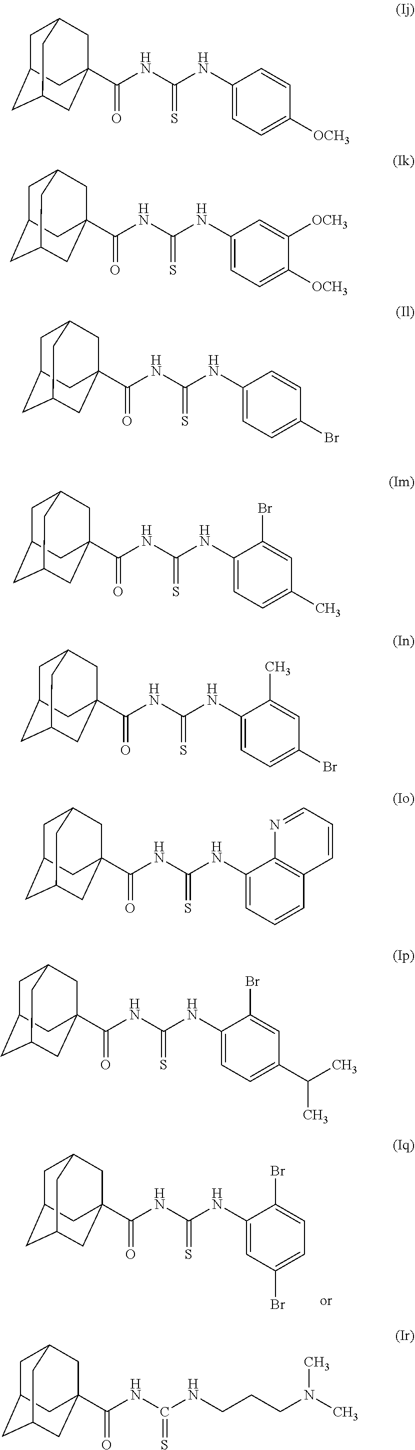

3. The compound of claim 1, wherein the compound is
N-((2-hydroxyphenyl)carbamothioyl)adamantane-1-carboxamide (Ia)
N-((3-hydroxyphenyl)carbamothioyl)adamantane-1-carboxamide (Ib)
N-((4-hydroxyphenyl)carbamothioyl)adamantane-1-carboxamide (Ic)
N-((3,5-dichloro-4-hydroxyphenyl)carbamothioyl)adamantane-1-carboxamide (Id)
N-((3-bromo-5-(trifluoromethyl)phenyl)carbamothioyl)adamantane-1-carboxamide (Ie)
N-((2-chloropyridin-3-yl)carbamothioyl)adamantane-1-carboxamide (If)
N-((3-sulfamoylphenyl)carbamothioyl)adamantane-1-carboxamide (Ig)
N-((2-chloro-5-nitrophenyl)carbamothioyl)adamantane-1-carboxamide (Ih)
N-((3-methoxyphenyl)carbamothioyl)adamantane-1-carboxamide (Ii)
N-((4-methoxyphenyl)carbamothioyl)adamantane-1-carboxamide (Ij)
N-((3,4-dimethoxyphenyl)carbamothioyl)adamantane-1-carboxamide (Ik)
N-((4-bromophenyl)carbamothioyl)adamantane-1-carboxamide (Il)
N-((2-bromo-4-methylphenyl)carbamothioyl)adamantane-1-carboxamide (Im)
N-((4-bromo-2-methylphenyl)carbamothioyl)adamantane-1-carboxamide (In)
N-(quinolin-8-ylcarbamothioyl)adamantane-1-carboxamide (Io)
N-((2-bromo-4-isopropylphenyl)carbamothioyl)adamantane-1-carboxamide (Ip)
N-((2,5-dibromophenyl)carbamothioyl)adamantane-1-carboxamide (Iq), or
N-((3-(dimethylamino)propyl)carbamothioyl)adamantane-1-carboxamide (Ir).

4. The compound of claim 1 for use as P2X receptor antagonist.

5. The compound of claim 1 for use in the treatment of inflammation, pain, osteoporosis, neurodegenerative disorders, spinal card injury, hypertension, or urinary incontinence.
